(19) United States
(12) Patent Application Publication
(10) Pub. No.: US 2013/0231580 A1
(43) Pub. Date: Sep. 5, 2013

(54) SEIZURE PREDICTION METHOD, MODULE AND DEVICE WITH ON-LINE RETRAINING SCHEME

(71) Applicants: Liang-Gee CHEN, Taipei (TW); Cheng-Yi CHIANG, Taipei (TW); Nai-Fu CHANG, Taipei (TW); Tung-Chien CHEN, Taipei (TW); Hong-Hui CHEN, Taipei (TW); Yun-Yu CHEN, Taipei (TW)

(72) Inventors: Liang-Gee CHEN, Taipei (TW); Cheng-Yi CHIANG, Taipei (TW); Nai-Fu CHANG, Taipei (TW); Tung-Chien CHEN, Taipei (TW); Hong-Hui CHEN, Taipei (TW); Yun-Yu CHEN, Taipei (TW)

(73) Assignee: NATIONAL TAIWAN UNIVERSITY, Taipei (TW)

(21) Appl. No.: 13/586,410

(22) Filed: Aug. 15, 2012

(30) Foreign Application Priority Data
Mar. 1, 2012 (TW) 101106823

(51) Int. Cl. A61B 5/048

(52) U.S. Cl. 600/544

(57) ABSTRACT

This invention is related to a seizure prediction method with an on-line retraining scheme. The seizure prediction method can self-learn the preictal and interictal waveforms of patients suffering from seizure with long-term brain signal monitoring, and can also distinguish the preictal waveforms from the interictal waveforms in real time to efficiently predict seizure. This invention also provides a seizure prediction module and a seizure prediction device to carry out the seizure prediction method.

This application claims the priority of Taiwan Patent Application No. 101106823, filed on Mar. 1, 2012. This invention is partly disclosed in oral presentation for Master Thesis on Sep. 3, 2011, entitled “Seizure Prediction Based on Classification of EEG Synchronization Patterns with On-line Retraining and Post-Processing Scheme” completed by Cheng-Yi Chiang, Nai-Fu Chang, Tung-Chien Chen, Hong-Hui Chen, and Liang-Gee Chen.

### FIELD OF THE INVENTION

The present invention relates to a seizure prediction method, and more particularly to a seizure prediction method with an on-line retraining scheme. This invention also provides a seizure prediction module and a seizure prediction device to carry out the seizure prediction method.

### BACKGROUND OF THE INVENTION

Epilepsy is one of the most common brain disorders in the clinic in the world. Epileptic seizures are caused due to excessive discharge of cerebral neurons associated with abnormal brain waves and behaviors. Abnormal brain waves and clinical symptoms are based on the discharge location of cortex, the pathway of transmission and the duration of the seizure. According to statistic, over 40 million people in the world suffer from epilepsy, wherein two-thirds of the patients achieve sufficient seizure control from medication or surgery. Besides, other patients have no best method of therapy, and thus must endure various inconveniences and dangers, and frequently worry about the uncertainty of the next seizure onset.

In the clinic, there is a plurality of methods for examining the disorder in brain, including revealing the structural images of brains by computerized tomography (CT), positron emission tomography (PET) or magnetic resonance imaging (MRI), and recording the variation of the electric signals of brains by electroencephalogram (EEG), wherein the tracing analysis method most suitable for a long period of time continuous detection to the patients is to record brain waveform by the EEG machine. The EEG machine is a type of non-invasive electric instrument, which firstly attaches a plurality of electrode patches on the head of a patient, transfers the detected electric signals to a transceiver by a connecting line, and then amplifies electric signals, filtrates and converts into digital signals for building up brain wave signals about activities of brain cells of the patient. A neurological physician can analyze, evaluate and trace the patients based on the brain wave recordings. Therefore, in the research field of brain waves, the analysis methods of brain waves are mainly used to examine disorders by signal processing or graphical identification, such as Fourier transforms (FT), Wavelet transforms (WT), Parametric modeling and Independent component analysis (ICA).

Recently, in the clinic, neurological physicians read brain waveforms of the epilepsy patients and observe that the epileptic patients having seizure a period of time later after particular spikes and sharp waves are performed, so that a theory of analyzing the particular variation of brain wave signals to predict the next preictal signal is proposed. Furthermore, the promotion of calculating ability of computing systems and the development of calculating software induce the researches in the biomedical engineering field to study the analysis and identification of brain wave signals, for the purpose of expecting to find out the best module for seizure prediction. At present, the method of predicting the preictal signals is generally the off-line training module in early-stage researches, wherein the off-line training module is a constant module that all of the training data are collected in advance. Owing to presume that brain wave signals are unchangeable and then to expect the module of the preictal signals to be stably maintained over a long period of time. However, the physical and psychological status, the severity degree of the preictal signals and different environmental variations during detection not only effect to brain wave signals, but also interfere with the validation of brain wave signals recording, resulting in reducing the accuracy of the prediction of the next seizure onset. Therefore, to apply the constant module of the off-line training method is insufficient to be a universal prediction module of the preictal signals for different patients.

As a result, it is necessary to provide a seizure prediction method, module and device with an on-line retraining scheme to solve the problems existing in the conventional technologies, as described above.

### SUMMARY OF THE INVENTION

A primary object of the present invention is to provide a seizure prediction method, module and device with an on-line retraining scheme, which is designed for solving the shortcoming existing in the conventional method of the constant off-line training module for predicting the preictal signals.

To achieve the above object, the present invention provides a seizure prediction method with an on-line retraining scheme, which comprises steps of:

recording brain wave signals continuously from an epilepsy patient by a brain wave recording unit, followed by receiving and transmitting the brain wave signals by a transceiver module;

extracting the brain wave signals as feature values by a processing module, aggregating these feature values into feature patterns, and then identifying if the feature patterns are an effective or ineffective preictal signal of seizure to define a classification value;

executing a post-processing analysis to the classification value by a post-process module, wherein an alarm signal is transmitted only if there are two or more consecutive classification values identified to be the effective preictal signals of seizure;

marking the current feature patterns and the past feature patterns stored within a predetermined time in the past by a marking device to obtain a preictal mark; and

executing an on-line retraining to the past feature patterns and the preictal mark thereof by a training unit of a classifier for renewing parameters of the classifier. For example, the training result can be used to renew parameters for operating a classifying unit of the classifier.

In one embodiment of the present invention, the brain wave recording unit continuously detects the variation of electric signals of brain from the epilepsy patient in a period of time, and comprises:

a plurality of electrode patches attached to a head of the epilepsy patient to be a detecting mediator;

a connecting line connected to the electrode patches for receiving an electric signals detected by the electrode patches;

a transceiver module connected to the connecting line for receiving and transmitting the electric signals; and

an EEG machine receiving the electric signals transmitted from the transceiver module, and filtrating the electric signals to transform into digital signals which are defined as brain wave signals.

In one embodiment of the present invention, the transceiver module is a wireless signal transceiver to wirelessly transmit the electric signals to the EEG machine.

In one embodiment of the present invention, the processing module comprises:

a feature pattern extracting unit periodically extracting the brain wave signals at a fixed interval, and stores the feature values to aggregate the feature values which are then transformed into low-dimensional feature patterns;

a feature pattern storing unit consecutively storing a plurality of the feature patterns;

the classifying unit of the classifier identifying and classifying the current feature patterns; and

the training unit of the classifier executing an on-line retraining to the stored feature patterns and the preictal mark thereof.

In one embodiment of the present invention, the processing module executes steps of:

periodically extracting the feature values of the brain wave signals at a fixed interval, consecutively aggregating a plurality of the feature values and then transforming into the feature patterns; and

identifying and classifying the feature patterns into the effective or ineffective preictal signals of seizure by the classifying unit of the classifier; then after a period of time, retraining the classifier by the training unit of the classifier according to marks provided by the marking device and a plurality of the feature patterns consecutively stored by the feature pattern storing unit, so as to obtain parameters which are then provided to the classifying unit of the classifier for enhancing the accuracy of classification.

In one embodiment of the present invention, the foregoing fixed interval is 5, 6, 7, 8, 9 or 10 minutes; and a cycle time of retraining is 30 minutes or less (such as 10 or 20 minutes), dependent on the calculation capability of the module.

In one embodiment of the present invention, the step of the post-processing analysis comprises: operating at least two of the classification values, wherein if an operation result determines that the classification values are two or more consecutive effective preictal signals of seizure, the alarm signal is transmitted to the epilepsy patient; and if the operation result determines that the classification values are not two or more consecutive effective preictal signals of seizure, the alarm signal is not transmitted.

In one embodiment of the present invention, the marking device is an auto-detecting marking device or a passive push-button marking device, and used to mark the current feature patterns as interictal signals of seizure, preictal signals of seizure or normal signals, and to mark the past feature patterns within the predetermined time in the past as the preictal signals of seizure or normal signals, wherein the predetermined time is a prediction period. For example, two hours is exemplified as the predetermined time, wherein if it assumes that a result caused by the auto-detecting marking device or the passive push-button marking device is marked as the interictal signal of seizure, and then a received feature pattern in the past two hours until now will be marked as the preictal signal, except for the feature patterns already marked as the interictal signal in the past. Alternatively, if it assumes that a result caused by the auto-detecting marking device or the passive push-button marking device is marked as the normal signal of seizure, and then the past feature patterns will not be marked, wherein the predetermined time can be one hour or two hours, but not limited thereto.

Furthermore, the present invention also provides a seizure prediction module with an on-line retraining scheme, detecting brain wave signals of an epilepsy patient and simultaneously predicting a preictal signal of seizure, wherein the seizure prediction module comprises:

a brain wave recording unit continuously recording brain wave signals of an epilepsy patient;

a transceiver module connected to the brain wave recording unit for receiving and transmitting the brain wave signals; and

a processing module connected to the transceiver module for transforming the received brain wave signals into feature patterns and identifying if the feature patterns are an effective preictal signal of seizure to generate a determination result which is then transmitted to a predetermined application.

Additionally, the present invention further provides a seizure prediction device with an on-line retraining scheme, wherein the seizure prediction device is an electrical product and comprises:

a control circuit for detecting, recording and storing brain wave signals of an epilepsy patient; and

a seizure prediction module connected to the control circuit for identifying the brain wave signals of the epilepsy patient to predict if the brain wave signals are preictal signals of seizure, wherein the seizure prediction module includes:

a transceiver module connected to the control circuit for receiving and transmitting the brain wave signals; and

a processing module connected to the transceiver module for transforming the received brain wave signals into feature patterns and identifying if the feature patterns are an effective preictal signal of seizure to generate a determination result which is then transmitted to a predetermined application.

In one embodiment of the present invention, the predetermined application is applied to an alarm device for transmitting an alarm signal to the epilepsy patient or a medical monitor in the medical organization, or applied to a medical treatment device for treating seizures of the epilepsy patient. Moreover, the alarm device can be a voice alarm device, a vibration alarm device, a light-emitting alarm device or a digital-display alarm device.

### DESCRIPTION OF THE DRAWINGS

FIG. 1 is a block diagram of a seizure prediction method with an on-line retraining scheme according to a preferred embodiment of the present invention;

FIG. 2 is a schematic view of a seizure prediction device with an on-line retraining scheme according to the preferred embodiment of the present invention;

FIG. 3 is a block diagram of a seizure prediction module with an on-line retraining scheme according to the preferred embodiment of the present invention;

FIG. 4 is a block diagram of a processing module of the seizure prediction method with an on-line retraining scheme according to the preferred embodiment of the present invention; and

FIG. 5 is an operational view of the processing module of the seizure prediction method with an on-line retraining scheme according to the preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

The present invention is to provide a seizure prediction method with an on-line retraining scheme, which is designed for resolving the shortcoming existing in the conventional method of the constant off-line training module for predicting the next seizure onset.

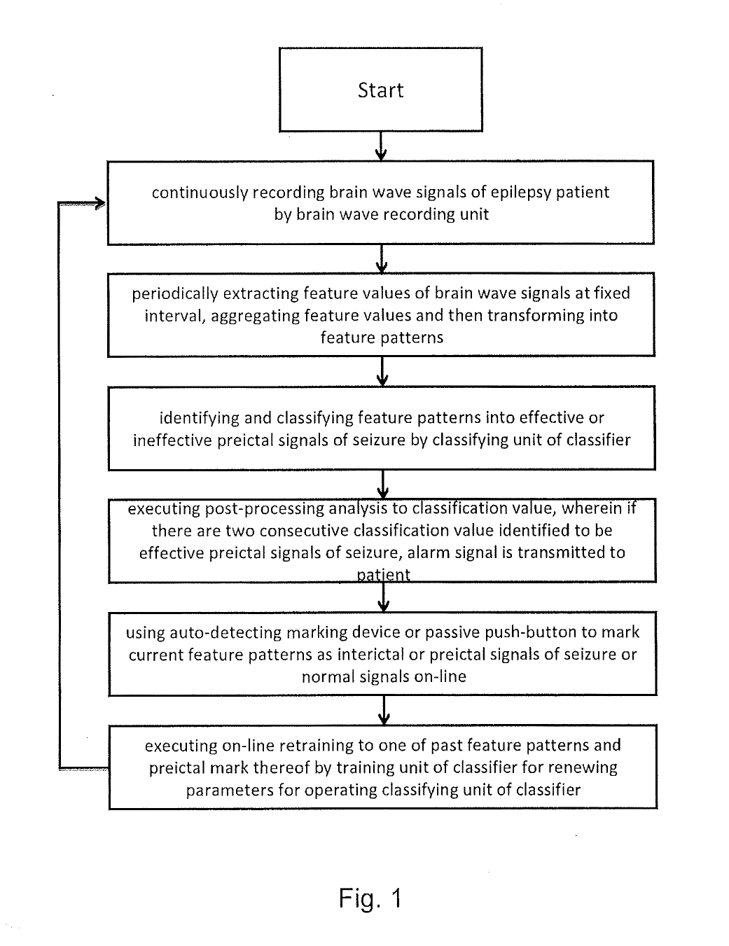

Referring to FIG. 1, a block diagram of a seizure prediction method with an on-line retraining scheme according to a preferred embodiment of the present invention is illustrated, wherein the seizure prediction method comprises steps of: continuously recording brain wave signals from an epilepsy patient by a brain wave recording unit, and receiving and transmitting the brain wave signals by a transceiver module; extracting the brain wave signals as feature values by a processing module, and aggregating these feature values into feature patterns, and then identifying if the feature patterns are an effective or ineffective preictal signal of seizure as a classification value; after this, executing a post-processing analysis to the classification value by a post-process module, wherein an alarm signal is transmitted only if there are two or more consecutive classification values identified to be the effective preictal signals of seizure; further marking the current feature patterns and the past feature patterns stored within a predetermined time in the past by a marking device to obtain a preictal mark; and executing an on-line retraining to the past feature patterns and the preictal mark thereof by a training unit of a classifier for renewing parameters for operating a classifying unit of the classifier, wherein the predetermined time can be 1 hour or 2 hours, but not limited thereto.

First, in the preferred embodiment of the present invention, brain wave signals from an epilepsy patient are continuously recorded by a brain wave recording unit for the purpose of setting up a particular database to the epilepsy patient by recording the brain wave signals from the epilepsy patient, and for selecting the best individual prediction module based on the particular database. The brain wave signals are received and transmitted by a transceiver module, which as a mediator. To prevent redundant connecting lines from causing inconvenience for the body and limbs of the epilepsy patient to move during recording continuously the brain wave signals of the epilepsy patient in a period of time, the transceiver module can be a wireless signal transceiver, which receives and transmits the brain wave signals to a processing module. Then, the processing module extracts the brain wave signals as feature values from the recorded brain wave signals, aggregates these feature values into feature patterns, and classifies the feature patterns as a classification value. The wireless signal transceiver can be a Bluetooth wireless signal transceiver, but not limited thereto.

In the preferred embodiment of the present invention, the brain wave signals from the epilepsy patient are continuously recorded in a period of time for being further modulated. Furthermore, for promoting the processing module to operate and analyze the brain wave signals, the processing module firstly extracts the brain wave signals as feature values by the feature pattern extracting unit, that is, to periodically extract one of the feature values as a representative of the brain wave signals at a fixed interval. Afterward, a plurality of continuous feature values is aggregated to be a feature pattern which is then converted into a low-dimensional feature pattern. Then, the low-dimensional feature pattern is identified into an effective or ineffective preictal signals by a classifying unit of a classifier. In the preferred embodiment of the present invention, the foregoing fixed interval can be 5, 6, 7, 8, 9 or 10 minutes, but not limited thereto.

Furthermore, in the preferred embodiment of the present invention, a post-process module executes a post-processing analysis to the classification value for the purpose of removing incorrect feature extractions caused due to external or personal factors to prevent from affecting the brain wave signals and generating an error in the classification value of the classifier. After this, the post-process module is set to decide if an operation result determines that the classification values are two or more consecutive effective preictal signals of seizure, in order to transmit an alarm signal to the epilepsy patient or a medical monitor as a pre-alarm; and if an operation result determines that the classification values are not two or more consecutive effective preictal signals of seizure, the alarm signal is not transmitted.

Then, to enhance the prediction precision of the preictal signals, the present invention further marks the current preictal signals by an auto-detecting marking device or a passive push-button marking device, for example, the auto-detecting marking device is used or a push-button is pushed by the epilepsy patient according to actual seizure states to confirm the preictal signals of the seizure pattern, so as to use the confirmation to mark and determine if a plurality of consecutive feature patterns are the preictal signals within the predetermined time in the past. Lastly, the training unit of the classifier is retrained according to the past feature patterns and the marks, and renewed parameters in the classifying unit of the classifier.

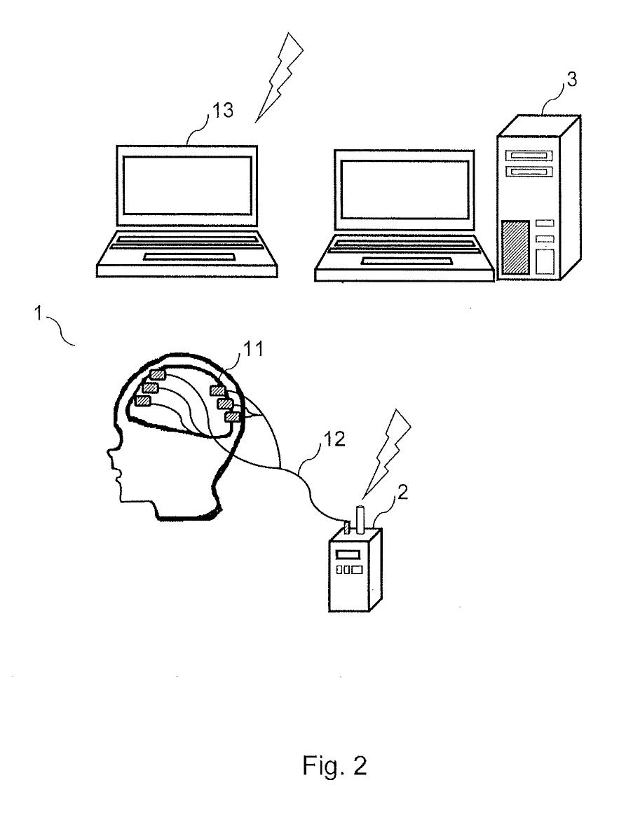

Referring to FIG. 2, a seizure prediction device with an on-line retraining scheme of the present invention is provided, wherein the brain wave recording unit 1 is used to continuously detect the variation of electric signals of brain from the epilepsy patient in a period of time. Firstly, a plurality of electrode patches 11 are attached to a head of the epilepsy patient to be a detecting mediator, wherein the attachment area of the electrode patches at least includes two parts corresponding to the prefrontal lobe of the frontal-head and the occipital lobe of the distal-head. Then, a connecting line 12 is connected to the electrode patches 11 for receiving electric signals detected by the electrode patches 11 and transmits the signals to a transceiver module 2. The transceiver module 2 is a wireless signal transceiver to wirelessly transmit the electric signals to the EEG machine. 13. Because the transmission between the transceiver module and the EEG machine is wirelessly achieved without connecting lines, the epilepsy patient can move within the allowed transmission range of the brain wave signals without affecting the record continuity of the brain wave signals. The wireless signal transceiver device is a Bluetooth wireless signal transceiver device, but not limited thereto.

Afterward, the data saved to the EEG machine 13 is used as a specific database of the epilepsy patient, and a processing module 3 is used to extract and transform the feature values to be the feature patterns, and then classifies the feature patterns by the classifier.

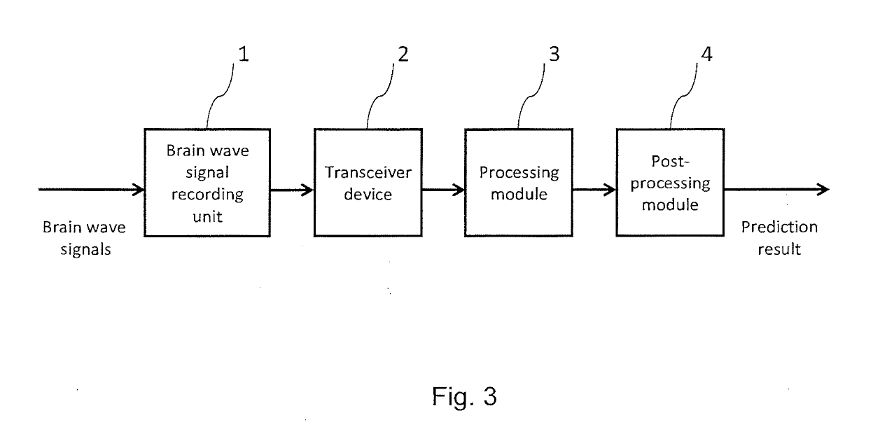

Referring to FIG. 3, a seizure prediction module with an on-line retraining scheme of the present invention is provided for detecting brain wave signals of an epilepsy patient to predict a preictal signal, wherein the seizure prediction module comprises: a brain wave recording unit 1 which continuously records brain wave signals of an epilepsy patient; a transceiver module 2 which is connected to the brain wave recording unit 1 for receiving and transmitting the brain wave signals; a processing module 3 which is connected to the transceiver module 2 for transforming the received the brain wave signals into feature patterns and classifies the feature patterns; and a post-process module 4 which is connected to the processing module 3 for operating at least two of the classification values, wherein if an operation result determines that the classification values are two or more consecutive effective preictal signals of seizure, the alarm signal is transmitted to the epilepsy patient, and if an operation result determines that the classification values are not two or more consecutive effective preictal signals of seizure, the alarm signal is not transmitted.

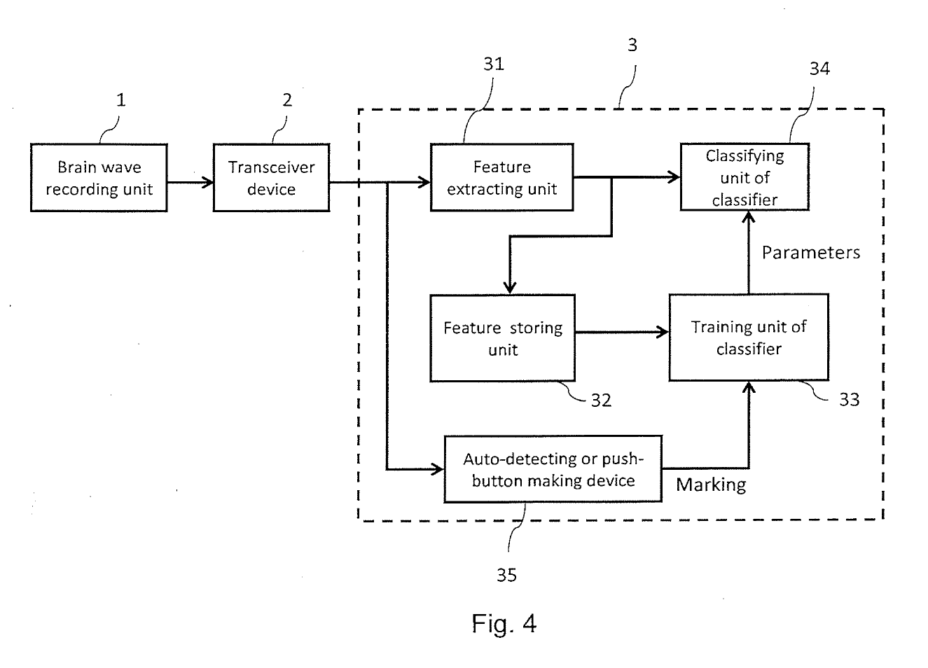

Referring to FIG. 4, a processing module of the seizure prediction method with an on-line retraining scheme of the present invention is provided, the processing module 3 comprises: a feature pattern extracting unit 31 which periodically extracts the brain wave signals at a fixed interval, and stores the feature values to aggregate the feature values which are then transforms into feature patterns; a feature pattern storing unit 32 which consecutively stores a plurality of the feature patterns; the training unit of the classifier 33 which retrains the classifying unit of the classifier and renews parameters of the classifying unit; the classifying unit of the classifier 34 which classifies current feature patterns is according to the renewed parameters; and an auto-detecting marking device (or a push-button device) 35 which is used to mark the past feature pattern. The processing module 3 is used to extract, transform and identify to the brain wave signals, wherein processing steps includes: periodically extracting the feature values of the brain wave signals at a fixed interval, consecutively aggregating a plurality of the feature values into a high-dimensional feature pattern which is then transformed into a low-dimensional feature pattern, identifying and classifying the feature patterns into the effective or ineffective preictal signal of seizure by the classifying unit of the classifier. As described above, the foregoing fixed interval can be 5, 6, 7, 8, 9 or 10 minutes and a cycle time of retraining is 30 minutes or less (such as 10 or 20 minutes, etc.), which is depend on the calculating ability of the module.

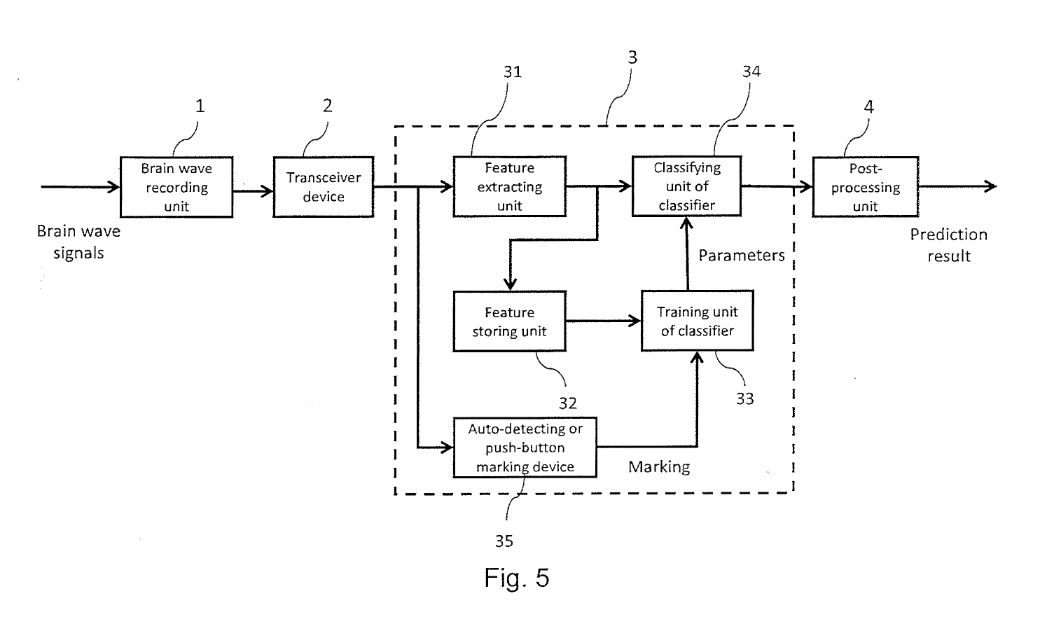

Referring to FIG. 5, a seizure prediction method with an on-line retraining scheme according to a preferred embodiment of the present invention is provided for detecting brain wave signals of an epilepsy patient and simultaneously predicting a preictal signal of seizure, wherein the seizure prediction module comprises: a brain wave recording unit 1 which continuously records brain wave signals from an epilepsy patient; a transceiver module 2 which is connected to the brain wave recording unit 1 for receiving and transmitting the brain wave signals; and a processing module 3 which is connected to the transceiver module 2 for transforming the received brain wave signals into feature patterns for determining a classification value of the feature patterns, followed by identifying and transmitting a determination result of the classification. In the embodiment, the processing module 3 comprises: a feature pattern extracting unit 31 which periodically extracts the brain wave signals at a fixed interval, and stores the feature values to aggregate the feature values which are then transforms into feature patterns; a feature pattern storing unit 32 which consecutively stores a plurality of the feature patterns; the training unit 33 of the classifier which is used for retraining the classifying unit of the classifier and renewing parameters of the classifying unit; the classifying unit 34 of the classifier which classifies current feature patterns according to the renewed parameters; and an auto-detecting marking device (or a push-button device) 35 which is used to mark the current feature patterns as preictal signals of seizure if necessary and to mark the past feature patterns within the predetermined time in the past as preictal signals of seizure. Then, a post-process module executes a post-processing analysis to the classification value, wherein if an operation result determines that the classification values are two or more consecutive effective preictal signals of seizure, the alarm signal is transmitted to the epilepsy patient.

Furthermore, the processing module 3 transmits a determination result to a predetermined application, wherein the predetermined application can be an alarm device which transmits an alarm signal to the epilepsy patient or a medical monitor in the medical organization, or applies to a medical treatment device for treating seizures to the epilepsy patient. Moreover, the alarm device can be a voice alarm device, a vibration alarm device, a light-emitting alarm device or a digital-display alarm device.

The disclosed features of the present invention are used to build up a specific database to the epilepsy patient according to the brain wave signals from the epilepsy patient, and to use the brain wave signals to train the classifier for the purpose of detecting the brain wave signals of an epilepsy patient and simultaneously predicting preictal signals of seizure in a period of time, followed by using the marking device and the training unit of the classifier to retain the classifier of the processing module, so as to improve the prediction module for enhancing the precision of predicting the preictal signals during the database is renewed. Therefore, the seizure prediction device with an on-line retraining scheme of the present invention can be used to transmit a highly precise preictal alarm signal to the epilepsy patient, and thus to improve the life quality of the daily life of the epilepsy patient.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A seizure prediction method with an on-line retraining scheme, comprising steps of:
continuously recording brain wave signals from an epilepsy patient by a brain wave recording unit, followed by receiving and transmitting the brain wave signals by a transceiver module;
extracting the brain wave signals as feature values by a processing module, aggregating these feature values into feature patterns, and then identifying if the feature patterns are an effective or ineffective preictal signal of seizure to define a classification value;
executing a post-processing analysis to the classification value by a post-process module, wherein an alarm signal is transmitted only if there are two or more consecutive classification value identified to be the effective preictal signals of seizure;
marking the current feature patterns and the past feature patterns stored within a predetermined time in the past by a marking device to obtain a preictal mark; and
executing an on-line retraining to the past feature patterns and the preictal mark thereof by a training unit of a classifier for renewing parameters for operating a classifying unit of the classifier.

2. The method according to claim 1, wherein the brain wave recording unit continuously detects the variation of electric signals of brain from the epilepsy patient in a period of time, and comprises:
a plurality of electrode patches attached to a head of the epilepsy patient to be a detecting mediator;
a connecting line connected to the electrode patches for receiving an electric signals detected by the electrode patches;
a transceiver module connected to the connecting line for receiving and transmitting the electric signals;
an EEG machine receiving the electric signals transmitted from the transceiver module, and filtrating the electric signals to transform into digital signals which are defined as brain wave signals.

3. The method according to claim 2, wherein the transceiver module is a wireless signal transceiver to wirelessly transmit the electric signals to the EEG machine.

4. The method according to claim 1, wherein the processing module comprises:
a feature pattern extracting unit periodically extracting the brain wave signals at a fixed interval, and stores the feature values to aggregate the feature values which are then transformed into low-dimensional feature patterns;
a feature pattern storing unit consecutively storing a plurality of the feature patterns;
the classifying unit of the classifier identifying and classifying the current feature patterns; and
the training unit of the classifier executing an on-line retraining to the stored feature patterns and the preictal mark thereof.

5. The method according to claim 4, wherein the processing module executes steps of:
periodically extracting the feature values of the brain wave signals at a fixed interval, consecutively aggregating a plurality of the feature values and then transforming into the feature patterns; and
identifying and classifying the feature patterns into the effective or ineffective preictal signals of seizure by the classifying unit of the classifier;
then after a period of time, retraining the classifier by the training unit of the classifier according to marks provided by the marking device and a plurality of the feature patterns consecutively stored by the feature pattern storing unit, so as to obtain parameters which are then provided to the classifying unit of the classifier for enhancing the accuracy of classification.

6. The method according to claim 4, wherein the fixed interval is 5, 6, 7, 8, 9 or 10 minutes; and a cycle time of retraining is 30 minutes or less.

7. The method according to claim 1, wherein the step of the post-processing analysis comprises:
operating at least two of the classification values;
if an operation result determines that the classification values are two or more consecutive effective preictal signals of seizure, the alarm signal is transmitted to the epilepsy patient; and
if the operation result determines that the classification values are not two or more consecutive effective preictal signals of seizure, the alarm signal is not transmitted.

8. The method according to claim 1, wherein the marking device is an auto-detecting marking device or a passive push-button marking device, and used to mark the current feature patterns as interictal signals of seizure, preictal signals of seizure or normal signals, and to mark the past feature patterns within the predetermined time in the past as preictal signals of seizure or normal signals, wherein the predetermined time is a prediction period.

9. A seizure prediction module with an on-line retraining scheme, detecting brain wave signals of an epilepsy patient and simultaneously predicting a preictal signal of seizure, comprising:
a brain wave recording unit persistently recording brain wave signals of an epilepsy patient;
a transceiver module connected to the brain wave recording unit for receiving and transmitting the brain wave signals; and
a processing module connected to the transceiver module for transforming the received brain wave signals into feature patterns and identifying if the feature patterns are an effective preictal signal of seizure to generate a determination result which is then transmitted to a predetermined application.

10. A seizure prediction device with an on-line retraining scheme, being an electrical product, comprising:
a control circuit detecting, recording and storing brain wave signals of an epilepsy patient; and
a seizure prediction module connected to the control circuit for identifying the brain wave signals of the epilepsy patient to predict if the brain wave signals are preictal signals of seizure, the seizure prediction module including:
a transceiver module connected to the control circuit for receiving and transmitting the brain wave signals; and
a processing module connected to the transceiver module for transforming the received brain wave signals into feature patterns and identifying if the feature patterns are an effective preictal signal of seizure to generate a determination result which is then transmitted to a predetermined application.
